(19) United States
(12) Patent Application Publication
(10) Pub. No.: US 2007/0196456 A1
(43) Pub. Date: Aug. 23, 2007

(54) Smart patch

(75) Inventors: John K. Stevens, Stratham, NH (US); Paul Waterhouse, Selkirk, ON (CA)

Correspondence Address: Oppedahl Patent Law Firm LLC - VAI, P.O. BOX 4850, FRISCO, CO, 80443-4850, US

(73) Assignee: VISIBLE ASSETS, INC., Missisauga, ON (CA)

(21) Appl. No.: 11/532,099

(22) Filed: Sep. 15, 2006

Related U.S. Application Data
(60) Provisional application No. 60/596,319, filed on Sep. 15, 2005.
(60) Provisional application No. 60/596,780, filed on Oct. 20, 2005.

(51) Int. Cl. A61N 1/30; A61L 15/16; G08C 19/16

(52) U.S. Cl. 424/448; 604/20; 340/870.2

(57) ABSTRACT

A combination of a patch and a low-frequency (inductive, LF) radiating radio transceiver tag, and antenna system, may be used to track and control electrophoretic/electro-osmotic transdermal drug delivery systems and provide fill data logs of use without complex belts that are worn by the patient or other patient-based attachments.

### CROSS-REFERENCE TO RELATED APPLICATIONS

This application claims priority from U.S. application Ser. No. 60/596,319 filed Sep. 15, 2005, and from U.S. application Ser. No. 60/596,780 filed Oct. 20, 2005, each of which is hereby incorporated herein by reference for all purposes.

### FIELD OF THE INVENTION

The invention relates generally to medicine patches, and relates more particularly to medicine patches that communicate by means of radio communication.

### BACKGROUND

As is known in the art, for some drugs (depending in part on the carrier used) electric current can increase the rate at which the drug enters the human body through the skin. Indeed for some drug-carrier combinations turning the current on and off will substantially stop and start the absorption of the drug into the human body through the skin.

Much energy has been poured into this sort of application for many years, and none of the efforts has thus far shown any success whatsoever. Some past investigators were convinced that 900 MHz was a good choice (prompted in part by its being in an unregulated ISM radio band) but proximity to the human body leads to poor RF propagation. Other past investigators were convinced that higher frequencies (around 13 gigahertz) were good choices, but these frequencies use up battery power all too quickly.

Passive RF systems (e.g. RFID systems) have drawbacks of being readable only if a reader is very nearby, typically on the order of inches.

The term “electrotransport” refers to the delivery of an agent (eg, a drug) through a membrane, such as skin, mucous membrane, or nails, by the application of an electric potential. The electrotransport process has been found to be useful in the transdermal administration of drugs including lidocaine hydrochloride, hydrocortisone, fluoride, penicillin, dexamethasone sodium phosphate, and many other drugs. Perhaps the most common use of electrotransport is in diagnosing cystic fibrosis by delivering pilocarpine salts iontophoretically.

Transdermal delivery of drugs has been in experimental use since the early 1900's (see U.S. Pat. No. 3,991,755: lontophoresis apparatus for applying local anesthetics. 1976) and has been in clinical use since the mid to late 1970's. Many improvements in pulse architecture U.S. Pat. No. 4,698,062: Medical device for pulsatile transdermal delivery of biologically active agents, 1987; U.S. Pat. No. 5,314,502: lontophoretic delivery device, 1994; U.S. Pat. No. 5,013,293: Pulsating transdermal drug delivery system, 1991, as well as methods for maximizing voltage as skin resistance increases U.S. Pat. No. 6,842,640: Electrotransport delivery device with voltage boosting circuit, 2005.

Controlled substances such as morphine, synthetic opioids, methadone, fentanyl and congeners of fentanyl such as sufentanil, alfentanil, lofentanil, carfentanil, remifentanil, have been administed to patients with great success by electro transport “patch” (U.S. Pat. No. 4,806,341: Transdermal absorption dosage unit for narcotic analgesics and antagonists and process for administration, 1989; U.S. Pat. No. 5,962,013: Monolithic matrix transdermal delivery system for administering molsidomine, 99; U.S. Pat. No. 5,962,013: Monolithic matrix transdermal delivery system for administering molsidomine, 99) This however has created a new set of issues not addressed by previous designs.

Since the patch content is a controlled substance detailed records are required of how much drug and administration time is required.

Different drug administered regimes may be required for different patents, so programmability may be required.

After the regime is completed some controlled substance may remain in the patch and disposal records and tracking of the used patch may be required. The potential for abuse of both new and used patch is high.

In some case the programmed regime may be incorrect or ineffective and it may be necessary to alter doses during administration of the drug.

It may be necessary to have information about current does provided up to specific date or time so changes may be made.

U.S. Pat. No. 5,149,538 (Misuse-resistive transdermal opioid dosage form 92) has addressed some of the issues by teaching that an antagonist may be mixed in small membrane breakable capsules with the active substance in such a way that when an individual tries to harvest the compound for illicit use the two will mix and be neutralized. This has the disadvantage that the agonist may be accidentally released and other dosage management issues are not addressed with this solution.

Radio Frequency Identity tags or RF-ID tags have a long history and, in recent times, RF-ID has become synonymous with “passive back-scattered transponders.” Passive transponders obtain power and a clock reference via a carrier and communicate by detuning an antenna often with a fixed pre-programmed ID. These tags are designed to replace barcodes and are capable of low power, two-way communications. Much of the patent literature and published literature surrounding these radio tags and RF-ID tags uses terminology that has not been well defined and can be confusing. We provide a glossary of terms and concepts used within this patent disclosure:

Radio Tag: Any telemetry system that communicates via magnetic (inductive communications) or electric radio communications to a base station or reader, or to another radio tag.

Passive Radio Tag: A radio tag that does not contain a battery.

Active Radio Tag: A radio tag that does contain a battery.

Transponder: A radio tag that requires a carrier from an integrator or base station to activate transmission or another function. The carrier is typically used to provide both power and a time-base clock.

Non-Radiating Transponder: A radio tag that may be active or passive and communicates via de-tuning or changing the tuned circuit of an antenna or coil; does not induce power into a transmitting antenna or coil.

Radiating Transponder: A radio tag or transponder that may be an active or passive tag, but communicates to the base station or interrogator by transmitting a radiated detectable electromagnetic signal by way of an antenna. The radio tag induces power into an antenna for its data transmission.

Back-Scattered Transponder: A radio tag that is identical to a non-radiating transponder; communicates by de-tuning an antenna and does not induce or radiate power in the antenna.

Transceiver: A radiating radio tag that actively receives digital data and actively transmits data by providing power to an antenna; may be active or passive.

Passive Transceiver: A radiating radio tag that actively receives and transmits digital data by providing power to an antenna, but does not have a battery and in most cases does not have a crystal or other time base source.

Active Transceiver: A radiating radio tag that actively receives digital data and actively transmits data by providing power to an antenna, and has a battery and in most cases a crystal or other internal time base source.

Inductive Field Mode: Uses low frequencies, 3-30 kHz VLF or the Myriametric frequency range, 30-300 kHz LF the Kilometric range, with some in the 300-3000 kHz MF or Hectometric range (usually under 450 kHz). Since the wavelength is so long at these low frequencies, over 99% of the radiated energy is magnetic, as opposed to a radiated electric field. Antennas are significantly (10 to 1000 times) smaller than ¼ wavelength or 1/10 wavelength, which would be required to efficiently radiate an electrical field.

Electric Field Mode: As opposed to the inductive mode radiation above, the electromagnetic mode uses frequencies above 3000 kHz in the Hectometric range, typically 8-900 MHz, where the majority of the radiated energy generated or detected may come from the electric field, and a ¼ or 1/10 wavelength antenna or design is often possible and utilized. The majority of radiated and detected energy is an electric field.

Many of the patents referenced do not make the distinctions outlined in the above glossary and may not have been (at the time) fully informed about functional significance related to the differences outlined above. For example, several of the early issued patents (e.g., U.S. Pat. No. 4,724,427, U.S. Pat. No. 4,857,893, U.S. Pat. No. 3,739,376, and U.S. Pat. No. 4,019,181) do not specify the frequency for the preferred embodiment, yet it has become clear that dramatic differences occur in performance and functional ability depending on the frequency. The frequency will change the radio tag's ability to operate in harsh environments, near liquids, or conductive materials, as well as the tag's range, power consumption and battery life.

The first reference to a radio tag in the patent literature was a passive radiating transponder described in U.S. Pat. No. 3,406,391: VEHICLE IDENTIFICATION SYSTEM, issued in 1968. The device was designed to track moving vehicles. U.S. Pat. No. 3,406,391 teaches that a carrier signal may be used to communicate to a radio tag in addition to providing power. The tags were powered using microwave frequencies and many sub-carrier frequencies were transmitted to the tag. The radio tag was programmed to pre-select several of the sub-carriers and provided an active re-transmission back when a sub-carrier corresponded to a set of preprogrammed bits in the tag. This multi-frequency approach limited data to about five to eight bits and the range of the device was limited to only a few inches.

U.S. Pat. No. 3,541,257, COMMUNICATION RESPONSE UNIT, issued in 1970, further taught that a digital address may be transmitted and detected to activate a radio tag. The radio tag may be capable of transmitting and receiving electromagnetic signals with memory, may work within a full addressable network, and has utility in many areas. Many other similar devices were described in the following years (e.g., The Mercury News, RFID pioneers discuss its origins, Sun, Jul. 18, 2004).

U.S. Pat. No. 3,689,885: INDUCTIVELY COUPLED PASSIVE RESPONDER AND INTERROGATOR UNIT HAVING MULTIDIMENSION ELECTROMAGNETIC FIELD CAPABILITIES, issued in 1972, and U.S. Pat. No. 3,859,624: INDUCTIVELY COUPLED TRANSMITTER-RESPONDER ARRANGEMENT, also issued in 1972, teach that a passive radiating digital radio tag may be powered and activated by induction using low frequencies (50 kHz), and transmit coded data modulated at a higher frequency (450 kHz) back to an integrator. They also show that the clock and 450 kHz-transmitting carrier from the radio tag may be derived from the 50 kHz induction power carrier. The inventors propose use of a ceramic filter to multiply the 50 kHz signal 9 times to get a frequency regenerate for the 450 kHz data out signal. These two patents also teach that steel and other conductive metals may de-tune the antennas and degrade performance. The ceramic filter required to increase the frequency from 50 kHz to a high frequency is, however, an expensive large external component, and phase locked loops or other methods commonly used to multiply a frequency would consume considerable power. These tags use the low frequency “power channel” to power the tag, serve as the time base for the tag, and finally as the trigger for the tag to transmit its ID. Thus, the power channel contains a single bit of on/off information.

U.S. Pat. No. 3,713,148: TRANSPONDER APPARATUS AND SYSTEM, issued in 1973, teaches that the carrier to the transponder may also transmit digital data and that the interrogation means (data input) may also be used to power the transponder. This patent also teaches that non-volatile memory may be added to store data that might be received and to track things like use and costs for tolls. The inventors do not specify or provide details on frequency or antenna configurations.

All devices referenced above rely on the antenna in radiating transceiver mode, where the power from the radio tag is actually “pumped” into a tuned circuit that includes a radiating antenna, which in turn produces an electromagnetic signal that can be detected at a distance by an interrogator.

U.S. Pat. No. 3,427,614: WIRELESS AND RADIOLESS (NONRADIANT) TELEMETRY SYSTEM FOR MONITORING CONDITIONS, issued in 1969, was the first to teach that the radio tag antenna may communicate simply by de-tuning the antenna rather than radiating power through the tuned antenna. The change in tuned frequency may be detected by a base station generating a carrier. This non-radiating mode reduces the power required to operate a tag and puts the detection burden on the base station. In effect, the radio tag's antenna becomes part of a tuned circuit created by the combination of the base station and a carrier. Any change in the radio tag's tuned frequency by any means can be detected by the base station's tuned carrier circuit. This is also often referred to as a back-scattered mode and is the basis for most modern RF-ID radio tags.

Many Electronic Article Surveillance (EAS) systems also function using this backscattered non-radiating mode (U.S. Pat. No. 4,774,504 1988, U.S. Pat. No. 3,500,373 1970, U.S. Pat. No. 5,103,234: Electronic article surveillance system, 1992), and most are also inductive frequencies. Many other telemetry systems in widespread use for pacemakers, implantable devices, and sensors in rotating centrifuges (U.S. Pat. No. 3,713,124: TEMPERATURE TELEMETERING APPARATUS, 1973) also make use of this back-scattered mode to reduce power consumption. U.S. Pat. No. 4,361,153 (Implant telemetry system, 1982) teaches that low frequencies (Myriametric) can transmit through conductive materials and work in harsh environments. Most of these implantable devices also use the back-scattered communication mode for communication to conserve battery power.

Accordingly, more recent and modern RF-ID tags are passive, back-scattered transponder tags and have an antenna consisting of a wire coil or an antenna coil etched or silk screened onto a PC board (e.g., see U.S. Pat. No. 4,857,893: Single chip transponder device, 1989; U.S. Pat. No. 5,682,143: Radio frequency identification tag, 1997). These tags use a carrier that is reflected back from the tag. The carrier is used by the tag for four functions:

1. The carrier contains the incoming digital data stream signal, in many cases the carrier only performs the logical function to turn the tag on/off and activate the transmission of its ID. In other cases, the data may be a digital instruction.

2. The carrier serves as the tag's power source. The tag receives a carrier signal from a base station and uses the rectified carrier signal to provide power to the integrated circuitry and logic on the tag.

3. The carrier serves as a clock and time base to drive the logic and circuitry within the integrated circuit. In some cases, the carrier signal is divided to produce a lower clock speed.

4. The carrier may also serve as a frequency and phase reference for radio communications and signal processing. The tag can use one coil to receive a carrier at a precise frequency and phase reference for the circuitry within the radio tag for communications back through a second coil to the reader/writer, making accurate signal processing possible (U.S. Pat. No. 4,879,756: Radio broadcast communication systems, 1989).

Thus, the main advantage of a passive back-scattered transponder is that it eliminates the battery as well as a crystal in LF tags. HF and UHF tags are unable to use the carrier as a time base because it would require high speed chips and power consumption would be too high. It is therefore generally assumed that a passive back-scattered transponder tag is less costly than an active or transceiver tag since it has fewer components and is less complex.

These modern non-radiating transponder back-scattered RF-ID tags typically operate at frequencies within the Part 15 rules of the FCC (Federal Communication Commission), between 10 kHz to 500 kHz (low frequency, LF, or ultra low frequency, ULF), 13.56 MHz (high frequency, HF), or 433 MHz (MHF) and 868/915 MHz or 2.2 GHz (ultra high frequency, UHF). The higher frequencies are typically chosen because they provide high bandwidth for communications, on a high speed conveyor for example, or where many thousands of tags must be read rapidly. In addition, it is generally believed that the higher frequencies are more efficient for transmission of signals and require much smaller antennas for optimal transmission. (It may be noted that a self-resonated antenna for 915 MHz can have a diameter as small as 0.5 cm and may have a range of tens of feet.)

However, the major disadvantage of the back-scattered mode radio tag is that it has limited power, limited range, and is susceptible to noise and reflections over a radiating active device. This is largely because the passive tag requires a minimum of 1 volt on its antenna to power the chip, not because of loss of communication signal. As a result, many back-scattered tags do not work reliably in harsh environments and require a directional “line of site” antenna.

One proposed method to extend the range of a passive back-scattered tag has been to add a thin, flat battery to the back-scattered tag so the power drop on the antenna is not the critical range limiting factor. However, since all of these tags use high frequencies, the tags must continue to operate in back-scattered mode to conserve battery life. The power consumed by any electronic circuit tends to be related to the frequency of operation. Thus, if a chip were to use an industry standard 280 mAh-capacity CR2525 Li cell (size of a quarter), we would expect, based solely on operating frequency, battery life to be:

Assumes 280 mAh Li Battery

| Frequency | | Current (μA) | Predicted life | units |
|---|---|---|---|---|
| 128 | kHz | 1 | 31.00 | years |
| 13.56 | MHz | 102 | 3.78 | months |
| 915 | MHz | 7031 | 1.66 | days |

Thus, most recent active RF-ID tags that may have a battery to power the tag circuitry are active tags and devices operating in the 13.56 MHz to 2.3 GHz frequency range, and also work as back-scattered transponders (U.S. Pat. No. 6,700,491: Radio frequency identification tag with thin-film battery for antenna, 2004; also see US 20040217865 A1: RFID tag 2004 for a detailed overview of issues). Because these tags are active backscattered transponders, they cannot work in an on-demand peer-to-peer network setting, or require line of sight antennas that provide a carrier that “illuminates” an area or zone or an array of carrier beacons.

Active radiating transceiver tags in the high frequency range (433 MHz) that can provide an on-demand peer-to-peer network of tags are available (e.g., SaviTag ST-654, U.S. Pat. No. 5,485,166: Efficient electrically small loop antenna with a planar base element, 1996) with full visibility systems described above (U.S. Pat. No. 5,686,902, U.S. Pat. No. 6,900,731). These tags do provide full functionality and so-called Real-Time Visibility, but they are expensive (over $100.00 US) and large (videotape-size, 6.25×2.125×1.125 inches) because of the power issues described above. They must also use replaceable batteries since even with a 1.5-inch by 6-inch Li battery, these tags are only capable of 2,500 reads and writes.

It is also generally assumed that HF or UHF passive back-scattered transponder radio tags will have a lower cost to manufacture than an LF passive back-scattered transponder because of the antenna. An HF or UHF tag can obtain a high Q, 1/10 wavelength antenna by etching or conductive silver silk screening the antenna geometry onto a flexi-circuit. An LF or ULF antenna cannot use either because the Q will be too low due to high resistance of the traces or silver paste. Therefore, LF and ULF tags must use wound coils made of copper.

Thus, in summary, a passive transponder tag has the potential to lower cost by eliminating the need for a battery as well as an internal frequency reference means. An active back-scattered transponder tag eliminates the extra cost of crystal while also providing for enhanced amplification of signals over a passive back-scattered transponder and enhanced range. In addition, it is also possible to use carrier reference to provide enhanced anti-collision methods to make it viable to read many tags within a carrier field (U.S. Pat. No. 6,297,734, U.S. Pat. No. 6,566,997, U.S. Pat. No. 5,995,019, U.S. Pat. No. 5,591,951). Finally, active radiating transceiver tags require large batteries and are expensive, perhaps costing up to hundreds of dollars.

Many publications and patents teach the advantages of using RF-ID tags for tracking products in warehouses, packages, etc. In some cases, passive transponders can be used but additional location and automated systems may be required for the base station (e.g., U.S. Pat. No. 6,705,522: Mobile object tracker, 2004). However, most literature now recognizes that a fully integrated peer-to-peer on-demand network approach, using active radio tags, has many functional advantages in these systems (U.S. Pat. No. 6,705,522: Mobile object tracker, 2004; U.S. Pat. No. 6,738,628: Electronic physical asset tracking, 2004; US 20020111819 A1: Supply chain visibility for real-time tracking of goods, 2002; U.S. Pat. No. 6,900,731: Method for monitoring and tracking objects, 2005; U.S. Pat. No. 5,686,902: Communication system for communicating with tags, 1997; and U.S. Pat. No. 4,807,140: Electronic label information exchange system, 1989). One of the major disadvantages of a passive non-radiating system is that it requires the use of handheld readers or portals to read tags and changes in process control (e.g., U.S. Pat. No. 6,738,628: Electronic physical asset tracking, 2004). A system that provides data without process change and without need for portal reads is more likely to be successful as a visibility system.

In previous disclosures, we have shown that the prior art has assumed low frequency tags are slow, short-ranged, and too costly. For example, both U.S. Pat. No. 5,012,236 and U.S. Pat. No. 5686902 discuss the short range issues associated with magnetic induction and low frequency tags. Because of the many apparent disadvantages of ULF and LF, the RF-ID frequencies are now recommended by many commercial (Item-Level Visibility In the Pharmaceutical Supply Chain: A Comparison of HF, UHF, and RFID Technologies, July 2004, Texas Instruments, Phillips Semiconductors, and TagSys Inc.) and government organizations (see Radio Frequency Identification Feasibility Studies and Pilot, FDA Compliance Policy HFC-230, Sec 400.210, November, 2004, recommend use of LF, HF or UHF), and standards associations (EPCglobal, web page tag specifications, January, 2005, note LF and ULF are excluded) do not mention or discuss the use of ULF as an option in many important retail applications. Many of the commercial organizations recommending these higher frequencies believe that passive and active radio tags in low frequencies are not suitable for any of these applications for reasons given above.

In addition, several commercial companies actually manufacture both ULF and LF radio tags (e.g., Texas Instruments and Philips Semiconductor, see Item-Level Visibility In the Pharmaceutical Supply Chain: A Comparison of HF, UHF, RFID Technologies, July 2004, Texas Instruments, Phillips Semiconductors, and TagSys Inc.), yet only recommend the use of 13.56 MHz or higher, again because of the perceived disadvantage of ULF and LF outlined above, and the many perceived advantages of HF, and UHF.

A detailed summary of the reasons that current LF radiating radio tags have not generally been considered for use in many modern applications is summarized below.

1. ULF is believed to have very short range since it uses largely inductive or magnetic radiance that drops off 1/d³, while far field HF and UHF drops off 1/d, where d is the distance from the source. Thus, the inductive or magnetic radiance mode of transmission will theoretically limit the distance of transmission, and that has been one of the major justifications for use of HF and UHF passive radio tags in many applications.

2. The transmission speed is inherently slow using ULF as compared to HF and UHF since the tag must communicate with low baud rates because of the low transmission carrier frequency.

3. Many sources of noise exist at these ULF frequencies from electronic devices, motors, florescent ballasts, computer systems, and power cables. Thus, ULF is often thought to be inherently more susceptible to noise.

4. Radio tags in this frequency range are considered more expensive since they require a wound coil antenna because of the requirement for many turns to achieve optimal electrical properties (maximum Q). In contrast, HF and UHF tags can use antennas etched directly on a printed circuit board. ULF would also have even more serious distance limitations with such an antenna.

5. Current networking methods used by high frequency tags, as used in HF and UHF, are impractical due to such low bandwidth of ULF tags described above in (3).

### SUMMARY OF THE INVENTION

A combination of a patch and a low-frequency (inductive, LF) radiating radio transceiver tag, and antenna system, may be used to track and control electrophoretic/electro-osmotic transdermal drug delivery systems and provide fill data logs of use without complex belts that are worn by the patient or other patient-based attachments.

We have disclosed the many non-obvious and unexpected advantages of low frequency, active radiating transceiver tags (WO 2006/085291 A2). They are especially useful for visibility and for tracking objects with large area loop antennas over other more expensive active radiating transponder HF/UHF tags (e.g., Savi ST-654). These LF tags will function in harsh environments, near water and steel, and may have full two-way digital communications protocol, digital static memory and optional processing ability, sensors with memory, and ranges of up to 100 feet. The active radiating transceiver tags can be far less costly than other active transceiver tags (many under one dollar), and often less costly than passive back-scattered transponder RF-ID tags, especially those that require memory and make use of EEPROM. These low frequency radiating transceiver tags also provide a high level of security since they have an on-board crystal than can provide a date-time stamp, making full AES encryption and one-time based pads possible. Finally, in most cases, LF active radiant transponder tags have a battery life of 10-15 years using inexpensive CR2525 Li batteries with 100,000 to 250,000 transmissions.

These active LF radiating transceiver tags may be used in a variety of applications; however, their intended use is within visibility networks for tracking assets in warehouses and moving vehicles, and they overcome many of the disadvantages of a passive back-scattered transponder tag system (U.S. Pat. No. 6,738,628: Electronic physical asset tracking, 2004). The tags may also be used for visibility networks for airline bags, evidence tracking, and livestock tracking, or in retail stores for tracking products.

We propose in this invention to provide address issues outlined above by adding a two-way low-frequency radiating radio tag to program, control and monitor the “patch”. We also provide additional functionality by adding a display and light emitting diodes (LEDs) to the patch as well as a small four-bit programmable processor so it may be monitored directly by a nurse or patient without any external devices. We also provide the option to release an antagonist under the control of the smart radio patch. The communication to the active radio tag may be via a large area loop antenna so that no human intervention is required. With the addition of a simple microprocessor it is possible to alter the dose and drug regime and also possible to deactivate the controlled substance at the end of the regime. Again this may be monitored and controlled remotely through a loop antenna without human intervention.

### DESCRIPTION OF THE DRAWINGS

FIG. 1: Prior art of a typical patch arrangement.

FIG. 2: Illustrates how the invention works. Item 8 is a crystal that provides accurate time base item 7 is a radio tag modem with optional memory and four bit processor item 9 is small loop antenna used for low frequency communications to a base station.

FIG. 3: Similar to FIG. 2 except a second reservoir has been added.

FIG. 4: Each patch may include optional LCD displays used to indicate status of the tag,

FIG. 5: The patch may be read using a base station and loop antenna similar to that described in U.S. Pat. No.4,937,586. The loops may be used as an area read around or in a room or bed or other localized area without any action on the part of the patient or health care worker.

FIG. 6: Block diagram of the device shown in FIG. 2 as item 4.

### DESCRIPTION OF THE INVENTION

In this application, we disclose a novel version of the active LF transponder that is combined with an active patch for delivering pharmaceuticals and in particular controlled substances. The radio tag can function in a full peer-to-peer network with any LF active radiating transponder as well as will large area loop antennas placed around a room or bed. This enables area read or “touchless” communication to and from the patch on a shelf or on a patient without any contact or process control change by the patient of staff.

Another unique aspect of the invention is the design of a low frequency active radio tag is not effected by “harsh environmental” factors commonly found with a transdermal delivery systems. Water or fluids associated with the patient or drug delivery system block UHF radio signals. Many of the drug delivery systems make use of aluminized flat batteries that can block both HF and UHF. By using a low frequency active transceiver there is no lose of signal as a result of liquids or an aluminized battery.

Another unique aspect of the invention is the fact the low frequency tag and its circuitry require minimal power since they operate at low clock frequencies. That makes it possible to use the same battery used by the patch for power to operate the chip(s) for many years when the tag may be in storage with no net lose of effective drug application.

Another unique aspect of the invention is the addition of an LCD display and LEDs. These may be used for a variety of different functions such as expiry date temperature maximums, current temperature, product identification, pick and put functions based on age or other criteria, automated recall if required, display of status. These may be manufactured using methods described in a previous disclosure (U.S. application Ser. No. 11/467,864, published as US publication number X) for embedding and sealing LCD's and LED'S and batteries at low temperatures.

Another unique aspect of the invention is that the identity of the patient, the patch lot number expiry date and use date may be automatically recorded, and provided as a record for use of controlled substances. This may be obtained from an area reader at an individual's home or clinic or hospital.

As may now be appreciated from the invention, for some prior art approaches the failure is due to unwise selection of operating frequencies.

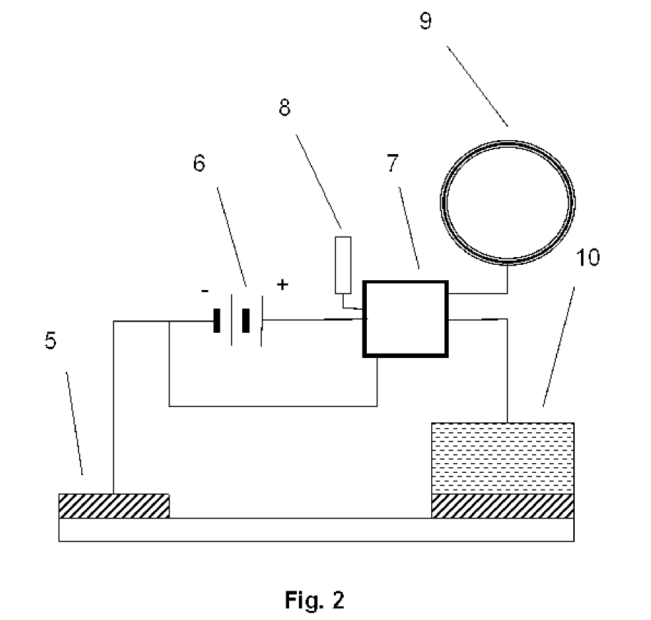

The smart patch shown in FIG. 2 is a patch providing a drug delivery system, for example morphine being delivered.

The patch includes an integrated circuit microcontroller and RF circuit 7, optionally a crystal 8, a battery 6, and a loop antenna 9. The RF circuitry operates typically in the range of 100 to 130 kilohertz, optionally up to perhaps 1 megahertz. Higher frequencies risk using up the battery 6 too quickly.

The selection of operating frequency, together with antenna dimensions consistent with a drug delivery patch (typically in the range of 1 by 1 inches to about three by three inches) permits “area reads” meaning that two-way radio communication is possible even from a room-sized distance. This means a reading/writing distance of at least a foot and preferably at least five feet.

The battery 6 is desirably a flat lithium battery.

A temperature sensor, omitted for clarity, may be attached to the controller 7 or may be integrally formed within the controller 7.

An electrically operated drug-delivery mechanism may be employed.

The crystal 8 permits accurate timekeeping and thus the microcontroller 11 can enter low-power mode most of the time, rising back to full-power mode only at particular times for purposes of finding out whether eternal equipment wishes to communicate with it.

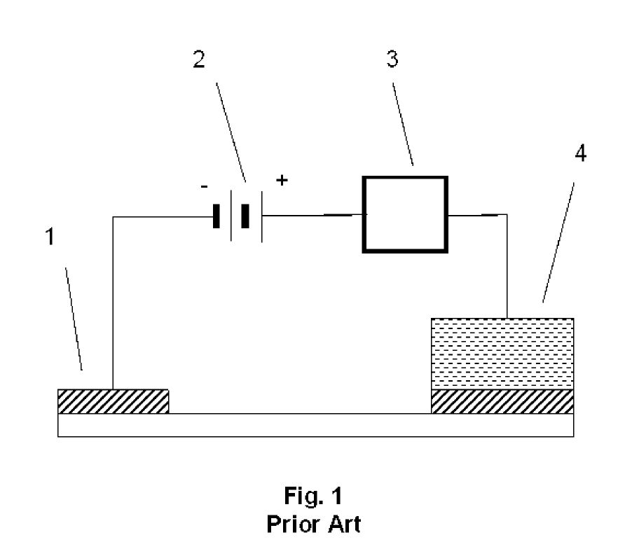

FIG. 1 shows a prior art of a typical patch arrangement. (U.S. Pat. No. 5,013,293) 1 is the negative electrode, 2 is typically a Li battery, 3 is circuit to manage current to 4 the drug reservoir. Other arrangements may use AC power to the two patches with ability to alter current based on the skin resistance.

FIG. 2 illustrates how the invention works. Item 8 is a crystal that provides accurate time base item 7 is a radio tag modem with optional memory and four bit processor. Item 9 is small loop antenna used for low frequency communications to a base station. The processor may also have optional sensors for temperature. The processor can be reprogrammed and controlled via the low frequency communication link controlled by item 7 and 9. Optional buttons may also be connected to item 7. Optional jog sensors may be placed on the processor to indicate activity. These can be simple low-cost sealed mercury switches or accelerometers. Data logs may be maintained in the processor and transmitted via the communications link.

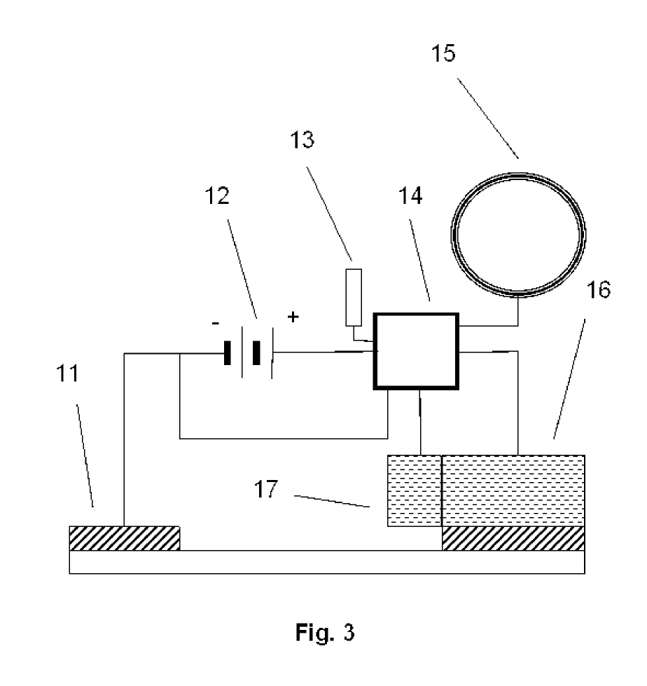

FIG. 3 is similar to FIG. 2 except a second reservoir 17 has been added. This second reservoir 17 may contain an agonist to the drug contained in compartment 16. At the end of a drug regime this may be activated to make any remaining drug harmless and not usable in compartment 16. This agonist is released by applying a voltage gradient between item 16 and item 17 and the agonist agent migrates across a conductive membrane to reservoir 16.

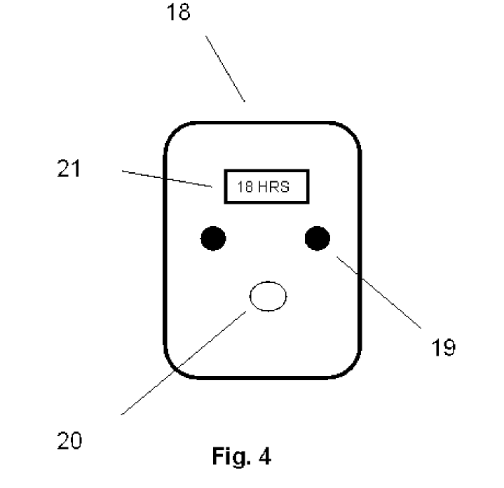

FIG. 4 shows that each patch may include optional LCD displays 21 used to indicate status of the tag, Light Emitting Diodes (LEDs) 19 are also used to indicate status or fault states of the patch. An optional button 20 may be added to indicate action from the patient. (e.g. Start or I am awake).

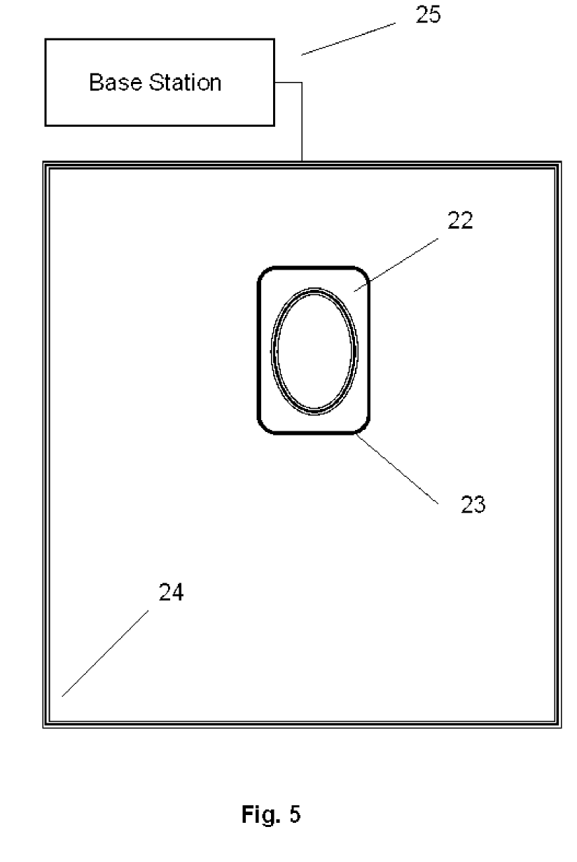

FIG. 5 shows how the patch may be read using a base station 25 and loop antenna 24 similar to that described in U.S. Pat. No. 4,937,586. The loops 24 may be used as an area read around or in a room or bed or other localized area without any action on the part of the patient or health care worker. The patch 23 may contain ID data, key information regarding the drug administered, and data logs associated with movement temperatures as well as dosage rate.

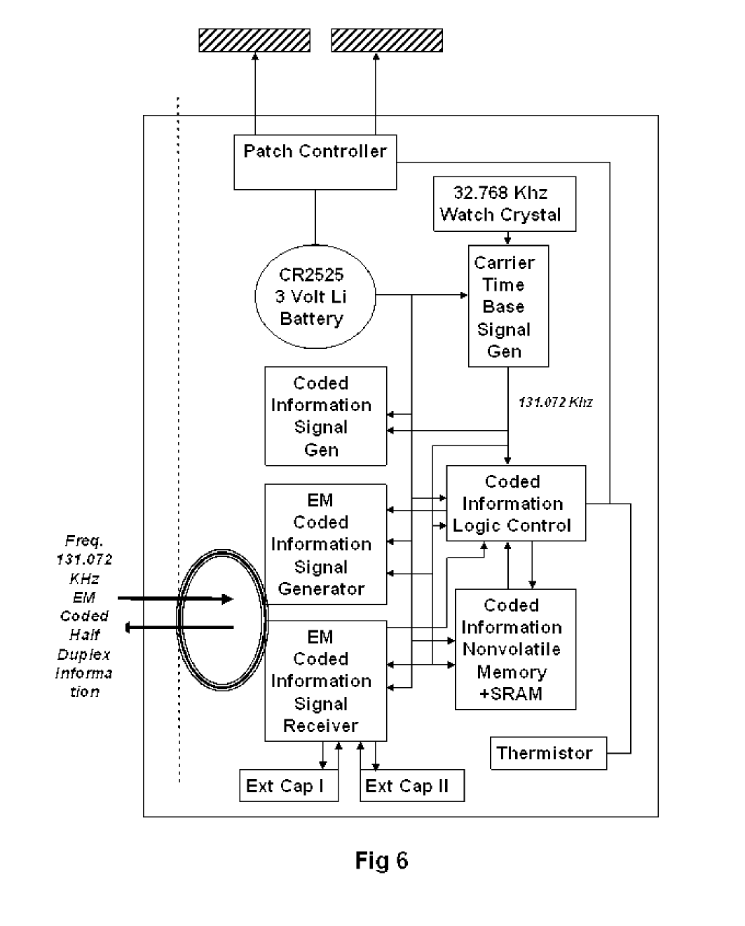

FIG. 6 is a block diagram of the device shown in FIG. 2 as item 4. The frequency is a harmonic of the crystal frequency 32.768 kHz, for example 131.072 kHz. The system may include sensors for temperature or movement (jog) and data logs may be kept in the memory.

Many benefits can flow from this apparatus. For example where the drug is a controlled substance (e.g. morphine) it is important to track each patch. This patch, due in large part to sensible selection of radio frequencies and other features mentioned above, is able to respond to “area reads” and thus the locations of the patches can be monitored in a hospital or other health-care environment, and indeed the patches can be more readily tracked as they enter or leave a secure area in a pharmacy.

The patch can optionally measure the ambient temperature on the side of the patch toward the skin. This permits monitoring whether the patch is in place on a patient's skin or whether it has been removed in which event the ambient temperature drops.

Those skilled in the art will have no difficulty devising myriad obvious improvements and variants of the invention without departing from the invention in any way, all of which are intended to be encompassed within the claims which follow.

1. A method for use with a plurality of medicine dispensing patches, each medicine dispensing patch comprising a microprocessor, a substrate with first and second sides, the first side disposed for contact with a patient and the second side toward the microprocessor, a loop antenna connected with the microprocessor, a battery connected with the microprocessor, a medicine dispensing reservoir adjacent the first side of the substrate, said medicine dispensing reservoir containing medicine, said medicine dispensing reservoir electrically connected with the microprocessor, said loop antenna and said microprocessor disposed to communicate by radio only at frequencies below 1 megahertz; the method performed with respect to a large antenna with area greater than five square feet, the method comprising the steps of:
transmitting a first message at a frequency below 1 megahertz by means of the large antenna;
receiving a response at a frequency below 1 megahertz from a first one of the plurality of medicine dispensing patches located in a vicinity of the large antenna;
transmitting a second message at a frequency below 1 megahertz by means of the large antenna;
receiving a response at a frequency below 1 megahertz from a second one of the plurality of medicine dispensing patches located in the vicinity of the large antenna;
removing the second one of the plurality of medicine dispensing patches from the vicinity of the large antenna;
transmitting the second message at a frequency below 1 megahertz by means of the large antenna;
failing to receive a response from the second one of the plurality of medicine dispensing patches; and
applying the second one of the plurality of medicine dispensing patches to an animal.

2. The method of claim 1 wherein the animal is a human being.

3. The method of claim 1 further comprising the step, performed after the failing step, of logging the event of failing to receive a response.

4. A method for use with a plurality of medicine dispensing patches, each medicine dispensing patch comprising a microprocessor, a substrate with first and second sides, the first side disposed for contact with a patient and the second side toward the microprocessor, a loop antenna connected with the microprocessor, a battery connected with the microprocessor, a medicine dispensing reservoir adjacent the first side of the substrate, said medicine dispensing reservoir containing medicine, said medicine dispensing reservoir electrically connected with the microprocessor, an agonist dispensing reservoir adjacent the medicine dispensing reservior, said agonist dispensing reservoir containing agonist, said agonist dispensing reservoir electrically connected with the microprocessor, said loop antenna and said microprocessor disposed to communicate by radio only at frequencies below 1 megahertz; the method performed with respect to a large antenna with area greater than five square feet, the method comprising the steps of:
transmitting a first message at a frequency below 1 megahertz by means of the large antenna;
receiving a response at a frequency below 1 megahertz from a first one of the plurality of medicine dispensing patches located in a vicinity of the large antenna;
transmitting a second message at a frequency below 1 megahertz by means of the large antenna;
receiving a response at a frequency below 1 megahertz from a second one of the plurality of medicine dispensing patches located in the vicinity of the large antenna;
removing the second one of the plurality of medicine dispensing patches from the vicinity of the large antenna;
transmitting the second message at a frequency below 1 megahertz by means of the large antenna;
failing to receive the second message at the second one of the plurality of medicine dispensing patches; and
passing current through the agonist reservoir and the medicine reservoir, whereby the agonist comes into contact with the medicine.

5. The method of claim 4 wherein the agonist deactivates the medicine.

6. The method of claim 4 wherein the medicine is a controlled substance.

7. The method of claim 6 wherein the medicine is morphine.

8. A medicine dispensing patch comprising:
a microprocessor;
a substrate with first and second sides, the first side disposed for contact with a patient and the second side toward the microprocessor;
a loop antenna connected with the microprocessor;
a battery connected with the microprocessor;
a medicine dispensing reservoir adjacent the first side of the substrate;
said medicine dispensing reservoir containing medicine;
said medicine dispensing reservoir electrically connected with the microprocessor;
said loop antenna and said microprocessor disposed to communicate by radio only at frequencies below 1 megahertz.

9. The medicine dispensing patch of claim 8, the patch further comprising:
a temperature sensor sensing a temperature;
the microprocessor responsive to received radio communications for transmitting information indicative of the sensed temperature.

10. The medicine dispensing patch of claim 8, the patch further comprising:
an agonist dispensing reservoir adjacent said medicine dispensing reservior;
the microcontroller electrically coupled with the agonist dispensing reservoir;
the microcontroller disposed, upon satisfaction of a predetermined event, to pass electrical current through the agonist dispensing reservoir and the medicine dispensing reservoir.

11. The medicine dispensing patch of claim 10, wherein the predetermined event is the passage of a predetermined interval of time.

12. The medicine dispensing patch of claim 11, wherein the predetermined event is the receipt of a predetermined message via the loop antenna 15.

13. The medicine dispensing patch of claim 8, further comprising a liquid-crystal display visible to a human user, the liquid-crystal display communicatively coupled with the microcontroller.

14. The medicine dispensing patch of claim 8, further comprising a light-emitting diode visible to a human user, the light-emitting diode communicatively coupled with the microcontroller.

15. The medicine dispensing patch of claim 8, further comprising a pushbutton visible to a human user, the push-button communicatively coupled with the microcontroller.

16. A method for use with a medicine dispensing patch, the medicine dispensing patch comprising a microprocessor, a substrate with first and second sides, the first side disposed for contact with a patient and the second side toward the microprocessor, a loop antenna connected with the microprocessor, a battery connected with the microprocessor, a medicine dispensing reservoir adjacent the first side of the substrate, said medicine dispensing reservoir containing medicine, said medicine dispensing reservoir electrically connected with the microprocessor, said loop antenna and said microprocessor disposed to communicate by radio only at frequencies below 1 megahertz; the method performed with respect to a large antenna with area greater than five square feet, the method comprising the steps of:
transmitting a first message at a frequency below 1 megahertz by means of the large antenna;
receiving a response at a frequency below 1 megahertz from the medicine dispensing patch located in a vicinity of the large antenna;
removing the medicine dispensing patch from the vicinity of the large antenna;
transmitting the first message at a frequency below 1 megahertz by means of the large antenna;
failing to receive a response from the medicine dispensing patch; and
applying the medicine dispensing patch to an animal.

17. The method of claim 16 wherein the animal is a human being.

18. The method of claim 16 further comprising the step, performed after the failing step, of logging the event of failing to receive a response.

19. A method for use with a medicine dispensing patch, the medicine dispensing patch comprising a microprocessor, a substrate with first and second sides, the first side disposed for contact with a patient and the second side toward the microprocessor, a loop antenna connected with the microprocessor, a battery connected with the microprocessor, a medicine dispensing reservoir adjacent the first side of the substrate, said medicine dispensing reservoir containing medicine, said medicine dispensing reservoir electrically connected with the microprocessor, an agonist dispensing reservoir adjacent the medicine dispensing reservior, said agonist dispensing reservoir containing agonist, said agonist dispensing reservoir electrically connected with the microprocessor, said loop antenna and said microprocessor disposed to communicate by radio only at frequencies below 1 megahertz; the method performed with respect to a large antenna with area greater than five square feet, the method comprising the steps of:
transmitting a first message at a frequency below 1 megahertz by means of the large antenna;
receiving a response at a frequency below 1 megahertz from the medicine dispensing patch located in a vicinity of the large antenna;
removing the medicine dispensing patch from the vicinity of the large antenna;
transmitting a second message at a frequency below 1 megahertz by means of the large antenna;
failing to receive the second message at the medicine dispensing patch; and
passing current through the agonist reservoir and the medicine reservoir, whereby the agonist comes into contact with the medicine.

20. The method of claim 19 wherein the agonist deactivates the medicine.

21. The method of claim 19 wherein the medicine is a controlled substance.

22. The method of claim 21 wherein the medicine is morphine.
